(19) United States
(12) Patent Application Publication
(10) Pub. No.: US 2024/0000684 A1
(43) Pub. Date: Jan. 4, 2024

(54) DYEING COMPOSITION BASED ON 2-GAMMA-HYDROXYPROPYL-PARA-PHENYLENEDIAMINE AND ON A PHOSPHORIC SURFACTANT

(71) Applicant: L'Oreal, Paris (FR)

(72) Inventors: Julie Blanc, SAINT OUEN (FR); Anne Sabbagh, SAINT OUEN (FR)

(73) Assignee: L'Oreal, Paris (FR)

(21) Appl. No.: 18/258,011

(22) PCT Filed: Dec. 16, 2021
(86) PCT No.: PCT/EP2021/086229

§ 371 Date: Jun. 16, 2023

(30) Foreign Application Priority Data
Dec. 17, 2020 (FR) 2013534

(51) Int. Cl. A61K 8/41; A61K 8/55; A61K 8/22; A61Q 5/10

(52) U.S. Cl. CPC A61K 8/411; A61K 8/55; A61K 8/22; A61Q 5/10; A61K 2800/4324; A61K 2800/882

(57) ABSTRACT

The present invention relates to a dyeing composition comprising 2-y-hydroxypropyl-p-phenylenediamine and at least one phosphoric surfactant. The phosphoric surfactant is of formula (I) in which: R₁, R₂ and R₃, which may be identical or different, represent a group chosen from: —a group —OM, in which M represents a hydrogen atom or an alkali metal; —a group —OR₄, in which R₄ represents a linear or branched C₁₂-C40 alkyl group, a linear or branched C₂-C₄₀ alkenyl group, a C₃-C₄₀ cyclic alkyl group, a C₃-C₄₀ cyclic alkenyl group, a C₅-C₄₀ aromatic group or a C₆-C₄₀ aralkyl group; and —an oxyalkylene group —(OCH₂CH₂)n(OCH₂CH(CH₃))mOR₄ in which R₄ is defined as above, n represents an integer ranging from 1 to 50 and m represents an integer ranging from 0 to 50; and given that at least one of the groups R₁, R₂ and R₃ is a group —OM and that at least one of the groups R₁, R₂ and R₃ is a group —OR₄ or —(OCH₂CH₂)n(OCH₂CH(CH₃))mOR₄. The present invention also relates to a process for dyeing keratin fibres, comprising the application of the composition according to the invention to the fibres. The invention also relates to the use of the combination 2-y-hydroxypropyl-para-phenylenediamine/phosphoric surfactant, for dyeing keratin fibres.

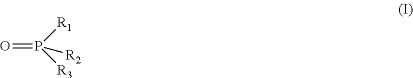

The present invention relates to a dyeing composition comprising 2-γ-hydroxypropyl-p-phenylenediamine and at least one phosphoric surfactant.

The present invention also relates to a process for dyeing keratin fibres, comprising the application of the composition according to the invention to the fibres. The invention also relates to the use of the combination 2-γ-hydroxypropyl-para-phenylenediamine/phosphoric surfactant, for dyeing keratin fibres.

It is known practice to dye keratin fibres and in particular human hair with dyeing compositions containing oxidation dye precursors, such as oxidation bases, especially ortho- or para-phenylenediamines (in particular 2-γ-hydroxypropyl-para-phenylenediamine), ortho- or para-aminophenols and heterocyclic compounds. These oxidation bases are colourless or weakly coloured compounds, which, when combined with oxidizing products, are able to produce coloured compounds by a process of oxidative condensation.

It is also known that the shades obtained with these oxidation bases may be varied by combining them with couplers or colour modifiers, the latter being chosen especially from aromatic meta-diaminobenzenes, meta-aminophenols, meta-diphenols and certain heterocyclic compounds such as indole compounds.

The variety of molecules used as oxidation bases and couplers allows a wide range of colours to be obtained.

“Permanent” dyeing is characterized by the use of oxidation dye precursor(s) (bases and/or couplers), such as 2-γ-hydroxypropyl-para-phenylenediamine, in the presence of oxidizing compound(s). In order to be considered as efficient dyeing, the latter needs to satisfy certain criteria. It must make it possible to obtain shades in the desired intensity with colour differences, between the end and the root of a same lock (also known as the selectivity), which are as small as possible.

The colouring must also be resistant over time and must not become degraded in the presence of external agents such as washing, light, bad weather, rubbing and perspiration.

However, the dyeing results obtained, in particular with a composition based on 2-γ-hydroxypropyl-para-phenylenediamine, are not always very satisfactory, especially in terms of selectivity, of colour buildup, of chromaticity, of intensity and/or of persistence, in particular with respect to successive shampoo washes, or of resistance to light or to perspiration.

There is thus a real need to develop new dyeing compositions based on 2-γ-hydroxypropyl-para-phenylenediamine, which make it possible to dye keratin fibres in a sparingly selective, intense, persistent, and chromatic manner, with good buildup of the colour, and which are capable of giving colourings that are resistant to the various attacking factors to which the fibres may be subjected, such as bad weather, washing and perspiration, and are also capable of resulting in good dyeing performance even after a period of storage.

These objectives are achieved by the present invention, one subject of which is notably a dyeing composition comprising:
(i) at least one oxidation base chosen from 2-γ-hydroxypropyl-para-phenylenediamine, salts thereof, solvates thereof, and mixtures thereof, and
(ii) at least one phosphoric surfactant chosen from phosphoric surfactants having the formula (I) below:

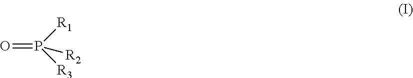

in which:
R₁, R₂ and R₃, which may be identical or different, represent a group chosen from:
   a group —OM, in which M represents a hydrogen atom or an alkali metal;
   a group —OR₄, in which R₄ represents a linear or branched C₁₂-C₄₀ alkyl group, a linear or branched C₂-C₄₀ alkenyl group, a C₃-C₄₀ cyclic alkyl group, a C₃-C₄₀ cyclic alkenyl group, a C₅-C₄₀ aromatic group or a C₆-C₄₀ aralkyl group; and
   an oxyalkylene group —(OCH₂CH₂)ₙ(OCH₂CH(CH₃))ₘOR₄ in which R₄ is defined as above, n represents an integer ranging from 1 to 50 and m represents an integer ranging from 0 to 50; and
given that at least one of the groups R₁, R₂ and R₃ is a group —OM and that at least one of the groups R₁, R₂ and R₃ is a group —OR₄ or —(OCH₂CH₂)ₙ(OCH₂CH(CH₃))ₘOR₄.

It has been noted that the composition according to the invention makes it possible to obtain better dyeing properties, especially in terms of selectivity.

It has also been noted that the colourings of the keratin fibres obtained by means of the composition of the invention exhibit good colouring buildup, good intensity, good chromaticity and good persistence.

The keratin fibre colourings obtained with the composition according to the invention are particularly persistent with respect to external agents (washing, light, bad weather, rubbing, perspiration), and especially persistent with respect to several shampoo washes.

A subject of the invention is also a process for dyeing keratin fibres, in particular human keratin fibres such as hair, comprising at least one step of applying to said fibres a composition according to the invention.

A subject of the invention is also the use of at least one oxidation base chosen from 2-γ-hydroxypropyl-para-phenylenediamine, salts thereof, solvates thereof, and mixtures thereof, in combination with at least one phosphoric surfactant of formula (I), for dyeing keratin fibres, in particular human keratin fibres such as the hair.

Other characteristics, aspects and advantages of the invention will become even more clearly apparent on reading the description and the example which follows.

In the present description, and unless otherwise indicated:
the expression “at least one” is equivalent to the expression “one or more” and can be replaced therewith;
the expression “between . . . and . . . ” is equivalent to the expression “ranging from . . . to . . . ” and can be replaced therewith, and implies that the limits are included;
for the purposes of the present invention, the expression “greater than” and respectively the expression “less than” are intended to mean an open range which is strictly greater, respectively strictly less, and therefore that the limits are not included;
the expression “fatty alcohol” denotes an alcohol comprising from 8 to 30 carbon atoms;
the expression “fatty acid” denotes an acid comprising from 8 to 30 carbon atoms;
the expression “fatty ether” denotes an ether comprising from 8 to 30 carbon atoms;
the expression “fatty ester” denotes an ester comprising from 8 to 30 carbon atoms;
the term “keratin fibres”, according to the present application, more particularly denotes human keratin fibres, and more preferentially the hair.

### 2-γ-Hydroxypropyl-para-phenylenediamine

The composition according to the invention comprises at least one oxidation base (i) chosen from 2-γ-hydroxypropyl-para-phenylenediamine, salts thereof, solvates thereof, and mixtures thereof.

2-γ-Hydroxypropyl-para-phenylenediamine is an oxidation dyeing base having the following chemical structure:

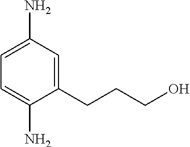 [SMILES: Nc1ccc(N)c(CCCO)c1]

2-γ-hydroxypropyl-para-phenylenediamine can also be present in the composition according to the invention in the form of a salt, for example an organic or mineral acid addition salt, and/or in the form of a solvate, for example a hydrate, or even in the form of a salt solvate.

More particularly, the composition according to the invention may comprise 2-γ-hydroxypropyl-para-phenylenediamine dihydrochloride.

Preferably, the total content of oxidation base(s) (i) present in the composition ranges from 0.001% to 20% by weight, more preferentially from 0.005% to 15% by weight, even more preferentially from 0.01% to 10% by weight, and even better still from 0.05% to 5% by weight, and even better still from 0.1% to 3% by weight, relative to the total weight of the composition.

Preferably, the total content of 2-γ-hydroxypropyl-para-phenylenediamine present in the composition ranges from 0.001% to 20% by weight, more preferentially from 0.005% to 15% by weight, even more preferentially from 0.01% to 10% by weight, and even better still from 0.05% to 5% by weight, and even better still from 0.1% to 3% by weight, relative to the total weight of the composition.

Additional Oxidation Bases

Preferably, the composition may also comprise one or more additional oxidation bases, other than the oxidation base(s) (i), and more preferentially other than 2-γ-hydroxypropyl-para-phenylenediamine, than salts thereof and than solvates thereof.

By way of example, the additional oxidation bases, other than the oxidation base(s) (i), can be chosen from para-phenylenediamines, bis(phenyl)alkylenediamines, para-aminophenols, ortho-aminophenols and heterocyclic bases, and the corresponding addition salts.

Among the para-phenylenediamines that may be mentioned are, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(3-hydroxyethyl)-para-phenylenediamine, 4-N,N-bis(3-hydroxyethyl)amino-2-methylaniline, 4-N,N-bis(p-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the corresponding addition salts with an acid.

Preference is in particular given, among the abovementioned para-phenylenediamines, to para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the corresponding addition salts with an acid.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(3-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the corresponding addition salts.

Among the para-aminophenols which are mentioned are, for example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the corresponding addition salts with an acid.

Among the ortho-aminophenols that may be mentioned, for example, are 2-aminophenol, 2-amino-5-methylphenol, 2-amino-6-methylphenol and 5-acetamido-2-aminophenol, and the corresponding addition salts.

Among the heterocyclic bases that may be mentioned are, for example, pyridine, pyrimidine and pyrazole derivatives.

Among the pyridine derivatives that may be mentioned are the compounds described, for example, in patents GB 1 026 978 and GB 1 153 196, for example 2,5-diaminopyridine, 2-(4-methoxyphenyl)amino-3-aminopyridine and 3,4-diaminopyridine, and the corresponding addition salts.

Other pyridine oxidation bases that are useful in the present invention are the 3-aminopyrazolo[1,5-a]pyridine oxidation bases or the corresponding addition salts described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-aminopyrazolo[1,5-a]pyridine and 2-(4-dimethylpiperazinium-1-yl)-3-aminopyrazolo[1,5-a]pyridine, and the corresponding addition salts.

More particularly, the oxidation bases that are useful in the present invention are chosen from 3-aminopyrazolo[1,5-a]pyridines and are preferably substituted on carbon atom 2 with:
a) a (di)(C₁-C₆)(alkyl)amino group, said alkyl group possibly being substituted with at least one hydroxyl, amino or imidazolium group;
b) an optionally cationic 5- to 7-membered heterocycloalkyl group comprising from 1 to 3 heteroatoms, optionally substituted with one or more (C₁-C₆)alkyl groups such as a di(C₁-C₄)alkylpiperazinium group; or
c) a (C₁-C₆)alkoxy group optionally substituted with one or more hydroxyl groups, such as a β-hydroxyalkoxy group, and the corresponding addition salts.

Among the pyrimidine derivatives that may be mentioned are the compounds described, for example, in patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, for instance 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the corresponding addition salts. Use may also be made of 4,5-diamino-1-(β-methoxyethyl)pyrazole.

A 4,5-diaminopyrazole will preferably be used and more preferentially still 4,5-diamino-1-(β-hydroxyethyl)pyrazole and/or a corresponding salt.

The pyrazole derivatives which may also be mentioned comprise diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one and 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a corresponding salt.

Use will preferably be made, as heterocyclic bases, of 4,5-diamino-1-(β-hydroxyethyl)pyrazole and/or 2,3-diamino-6,7-dihydro-1H,5H-pyrazolo[1,2-a]pyrazol-1-one and/or 2-β-hydroxyethoxy-3-aminopyrazolo[1,5-a]pyridine and/or a corresponding salt.

More preferentially, the additional oxidation base(s), other than the oxidation base(s) (i), are chosen from para-phenylenediamine, para-toluenediamine, para-aminophenol, 2-β-hydroxyethyl-para-phenylenediamine, N,N-bis(β-hydroxyethyl)-para-phenylenediamine, 4,5-diamino-1-(β-hydroxyethyl)pyrazole, 2,3-diamino-6,7-dihydro-1H,5H-pyrazolo[1,2-a]pyrazol-1-one, 2-β-hydroxyethoxy-3-aminopyrazolo[1,5-a]pyridine, addition salts thereof, solvates thereof, and mixtures thereof.

In one particular embodiment of the invention, the composition is free from oxidation bases chosen from para-phenylenediamine, para-toluenediamine, addition salts thereof, and solvates thereof.

Preferably, when the composition comprises at least one additional oxidation base other than the oxidation base(s) (i), the total content of additional oxidation base(s) ranges from 0.001% to 10% by weight, more preferentially 0.005% to 5% by weight, relative to the total weight of the composition according to the invention.

Oxidation Couplers

Preferably, the composition may also comprise one or more oxidation couplers.

By way of example, the oxidation couplers can be chosen from meta-phenylenediamines, meta-aminophenols, meta-diphenols, naphthalene-based coupling agents and heterocyclic coupling agents, and also their geometrical or optical isomers, their tautomers, their corresponding addition salts or their solvates according to the invention.

Mention may be made, for example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole and 6-methylpyrazolo[1,5-a]benzimidazole, 2-methyl-5-aminophenol, 2-amino-5-ethylphenol, hydroxyethyl-3,4-methylenedioxyaniline, 5-N-(β-hydroxyethyl)amino-2-methylphenol, 3-aminophenol, 3-amino-2-chloro-6-methylphenol, 2-([3-amino-4-methoxyphenyl]amino)ethanol, and the corresponding addition salts with an acid.

More preferentially, the oxidation coupler(s) are chosen from meta-phenylenediamines, meta-aminophenols, meta-diphenols, naphthalene-based coupling agents, heterocyclic coupling agents, addition salts thereof, solvates thereof, and mixtures thereof; and even more preferentially from 2-amino-5-ethylphenol, hydroxyethyl-3,4-methylenedioxyaniline,1,3-dihydroxybenzene, 1,3-dihydroxy-2-methylbenzene, 3-aminophenol, 6-hydroxybenzomorpholine, 5-N-(β-hydroxyethyl)amino-2-methylphenol, 2,4-diamino-1-(β-hydroxyethyloxy)benzene, 2-methyl-5-aminophenol, 6-hydroxyindole, 4-chloro-1,3-dihydroxybenzene, 2-amino-3-hydroxypyridine, 3-amino-2-chloro-6-methylphenol, α-naphthol, 2-[3-amino-4-methoxyphenyl]amino)ethanol, addition salts thereof, solvates thereof, and mixtures thereof.

In one particular embodiment of the invention, the composition is free from oxidation couplers chosen from resorcinol, 2-methylresorcinol and 4-chlororesorcinol, addition salts thereof, and solvates thereof.

Preferably, when the composition comprises at least one oxidation coupler, the total content of oxidation coupler(s) ranges from 0.001% to 10% by weight, more preferentially 0.005% to 5% by weight, relative to the total weight of the composition according to the invention.

In general, the addition salts of oxidation bases or of oxidation couplers that can be used in the context of the invention are chosen from the addition salts with an acid, such as the hydrochlorides, hydrobromides, sulfates, citrates, succinates, tartrates, lactates, tosylates, benzenesulfonates, phosphates and acetates.

Phosphoric Surfactants

The composition according to the invention comprises at least one phosphoric surfactant of formula (I).

For the purposes of the present invention, the term “phosphoric surfactant” means a surfactant of which the polar part comprises at least one phosphorus atom.

The phosphoric surfactants of formula (I) have the following structure:

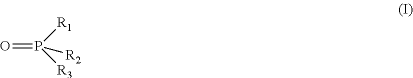

in which:
R₁, R₂ and R₃, which may be identical or different, represent a group chosen from:
   a group —OM, in which M represents a hydrogen atom or an alkali metal, such as Na, Li or K, preferably Na or K;
   a group —OR₄, in which R₄ represents a linear or branched C₁₂-C₄₀ alkyl group, preferably a C₁₂-C₂₀ alkyl group, more preferentially a C₁₆ or C₁₈ alkyl group, a linear or branched C₂-C₄₀ alkenyl group, preferably a C₁₂-C₂₀ alkenyl group, more preferentially a C₁₆ or C₁₈ alkenyl group, a C₃-C₄₀ cyclic alkyl group, a C₃-C₄₀ cyclic alkenyl group, a C₅-C₄₀ aromatic group or a C₆-C₄₀ aralkyl group; and
   an oxyalkylene group —(OCH₂CH₂)ₙ(OCH₂CH(CH₃))ₘOR₄ in which R₄ is defined as above, n represents an integer ranging from 1 to 50 and m represents an integer ranging from 0 to 50,
given that at least one from among R₁, R₂ and R₃ is a group —OM and that at least one from among R₁, R₂ and R₃ is a group —OR₄ or —(OCH₂CH₂)ₙ(OCH₂CH(CH₃))ₘOR₄.

Preferably, the phosphoric surfactant(s) are chosen from polyoxyalkylenated fatty alcohol phosphates containing from 12 to 20 carbon atoms and from 1 to 50 mol of alkylene oxide chosen from ethylene oxide and propylene oxide, and non-polyoxyalkylenated fatty alcohol dialkyl phosphates containing from 12 to 22 carbon atoms, and mixtures thereof.

The alkyl group of the polyoxyalkylenated fatty alcohol or of the non-polyoxyalkylenated fatty alcohol may be linear or branched, and saturated or unsaturated.

More preferentially, the phosphoric surfactant(s) are chosen from polyoxyalkylenated fatty alcohol phosphates containing from 12 to 20 carbon atoms and from 1 to 50 mol of alkylene oxide chosen from ethylene oxide and propylene oxide. Even more preferentially, the phosphoric surfactant(s) are chosen from polyoxyethylenated fatty alcohol phosphates containing from 12 to 20 carbon atoms and from 1 to 50 mol of ethylene oxide.

According to one particular embodiment of the invention, the composition may comprise a combination of at least one oxyalkylenated phosphoric surfactant and of at least one non-oxyalkylenated phosphoric surfactant.

More preferentially, according to this embodiment, the combinations of phosphoric surfactants may be chosen from a combination of ceteth-10 phosphate and dicetyl phosphate, a combination of ceteth-20 phosphate and dicetyl phosphate, and a combination of oleth-5 phosphate and dioleyl phosphate.

As product comprising ceteth-10 phosphate, mention may be made of Crodafos CES or Crodafos CES-PA sold by Croda. As product comprising ceteth-20 phosphate, mention may be made of Crodafos CS-20 Acid sold by Croda. As product comprising oleth-5 phosphate, mention may be made of Crodafos HCE sold by Croda.

More particularly preferably, the phosphoric surfactant(s) are chosen from ceteth-10 phosphate, dicetyl phosphate, ceteth-20 phosphate, oleth-5 phosphate, dioleyl phosphate, salts thereof, and mixtures thereof.

In addition, most particularly preferably, the phosphoric surfactant(s) are chosen from ceteth-10 phosphate, ceteth-20 phosphate, oleth-5 phosphate, salts thereof, and mixtures thereof; and even better still from ceteth-10 phosphate, salts thereof, and mixtures thereof.

Preferably, the total content of phosphoric surfactant(s) of formula (I) present in the composition according to the invention ranges from 0.01% to 15% by weight, more preferentially from 0.05% to 10% by weight, even more preferentially from 0.075% to 5% by weight, better still from 0.1% to 2% by weight, and even better still from 0.5% to 1% by weight, relative to the total weight of the composition.

Preferably, the weight ratio of the total content of phosphoric surfactant(s) of formula (I) on the one hand, to the total content of 2-γ-hydroxypropyl-para-phenylenediamine on the other hand, is greater than or equal to 1, preferentially greater than or equal to 1.5, more preferentially greater than or equal to 2, even more preferentially greater than or equal to 2.5.

According to a preferred embodiment of the invention, the weight ratio of the total content of phosphoric surfactant(s) of formula (I) on the one hand, to the total content of 2-γ-hydroxypropyl-para-phenylenediamine on the other hand, ranges from 1 to 10, more preferentially from 1.5 to 10, even more preferentially from 2 to 8 and better still from 2.5 to 5.

Additional Surfactants

Preferably, the composition may also comprise one or more additional surfactants, other than the phosphoric surfactants.

More preferentially, the composition according to the invention also comprises one or more non-ionic surfactants, other than phosphoric surfactants.

By way of example, the non-ionic surfactant(s) may be chosen from C₈-C₃₀ alcohols, C₈-C₃₀ alpha-diols and (C₁-C₂₀)alky phenols, these compounds also being polyethoxylated and/or polypropoxylated and/or polyglycerolated, the number of ethylene oxide and/or propylene oxide groups possibly ranging from 1 to 200, and the number of glycerol groups possibly ranging from 2 to 30.

Mention may also be made of condensates of ethylene oxide and of propylene oxide with fatty alcohols; polyethoxylated fatty amides preferably containing from 2 to 30 ethylene oxide units, polyglycerolated fatty amides including on average from 1 to 5, and in particular from 1.5 to 4, glycerol groups; ethoxylated fatty acid esters of sorbitan preferably containing from 2 to 40 ethylene oxide units, fatty acid esters of sucrose, polyoxyalkylenated and preferably polyoxyethylenated fatty acid esters containing from 2 to 150 mol of ethylene oxide, including oxyethylenated plant oils, N—(C₆₋₂₄ alkyl)glucamine derivatives, amine oxides such as (C₁₀₋₁₄ alkyl)amine oxides or N—(C₁₀₋₁₄ acyl)aminopropylmorpholine oxides.

Mention may also be made of non-ionic surfactants of alkyl(poly)glycoside type, represented notably by the following general formula:

R₁O—(R₂O)ₜ-(G)ᵥ

in which:
R₁ represents a linear or branched alkyl or alkenyl radical including 6 to 24 carbon atoms and notably 8 to 18 carbon atoms, or an alkylphenyl radical of which the linear or branched alkyl radical includes 6 to 24 carbon atoms and notably 8 to 18 carbon atoms,
R₂ represents an alkylene radical including 2 to 4 carbon atoms,
G represents a sugar unit including 5 to 6 carbon atoms,
t denotes a value ranging from 0 to 10 and preferably from 0 to 4,
v denotes a value ranging from 1 to 15 and preferably from 1 to 4.

Preferably, the alkyl(poly)glycoside surfactants are compounds of the formula described above in which:
R₁ denotes a linear or branched, saturated or unsaturated alkyl radical including from 8 to 18 carbon atoms,
R₂ represents an alkylene radical including 2 to 4 carbon atoms,
t denotes a value ranging from 0 to 3 and preferably equal to 0,
G denotes glucose, fructose or galactose, preferably glucose;
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C₈/C₁₆ alkyl (poly)glucosides of 1-4 type, and in particular decyl glucosides and caprylyl/capryl glucosides, are very particularly preferred.

Among the commercial products, mention may be made of the products sold by the company Cognis under the names Plantaren® (600 CS/U, 1200 and 2000) or Plantacare® (818, 1200 and 2000); the products sold by the company SEPPIC under the names Oramix CG 110 and Oramix® NS 10; the products sold by the company BASF under the name Lutensol GD 70, or the products sold by the company Chem Y under the name AG10 LK.

Preferably, use is made of C₈/C₁₆ alkyl (poly)glycosides of 1-4 type, in particular as an aqueous 53% solution, such as those sold by Cognis under the reference Plantacare® 818 UP.

Preferentially, the non-ionic surfactants are chosen from (C₆-C₂₄ alkyl)(poly)glycosides, and more particularly (C₈-C₁₈ alkyl)(poly)glycosides, ethoxylated C₈-C₃₀ fatty acid esters of sorbitan, polyoxyalkylenated C₈-C₃₀ fatty alcohols and polyoxyalkylenated C₈-C₃₀ fatty acid esters, these compounds preferably also containing from 2 to 200 mol of alkylene oxide, and mixtures thereof.

Preferably, when they are present, the composition according to the invention comprises said additional surfactant(s), other than the phosphoric surfactants, in an amount ranging from 0.01% to 20% by weight, more preferentially ranging from 0.5% to 15% by weight, even more preferentially from 1% to 15% by weight, and even better still from 3% to 10% by weight, relative to the total weight of the composition.

Preferably, when they are present, the composition according to the invention comprises said additional non-ionic surfactant(s), other than the phosphoric surfactants, in an amount ranging from 0.01% to 20% by weight, more preferentially ranging from 0.5% to 15% by weight, even more preferentially from 1% to 15% by weight, and even better still from 3% to 10% by weight, relative to the total weight of the composition.

Non-Silicone Fatty Substances

Preferably, the composition may also comprise one or more non-silicone fatty substances.

For the purposes of the present invention, the term “fatty substance” is intended to mean an organic compound that is insoluble in water at ordinary temperature (25° C.) and at atmospheric pressure (760 mmHg or 1.013×10⁵) (solubility in water of less than 5 g of fatty substance per ml of water, preferably less than 1 g/ml of water and even more preferentially less than 0.1 g/ml of water).

The non-silicone fatty substances (i.e. the fatty substances not comprising any silicon atoms in their structure) have in their structure at least one hydrocarbon-based chain including at least 8 carbon atoms. In addition, the non-silicone fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

The non-silicone fatty substances of the invention do not contain any salified carboxylic acid groups.

In addition, the non-silicone fatty substances of the invention are not (poly)oxyalkylenated or (poly)glycerolated ethers.

The non-silicone fatty substances are different from the surfactants described above.

By way of example, the nonsilicone fatty substance(s) that can be used may be chosen from fatty alcohols; mineral, plant or animal oils; liquid fatty esters; liquid hydrocarbons, and mixtures thereof.

The fatty alcohols may be linear or branched. They preferably comprise 8 to 30 carbon atoms; they may be saturated or unsaturated.

The saturated fatty alcohols are preferably branched. They can optionally comprise, in their structure, at least one aromatic or non-aromatic ring. Preferably, they are acyclic. More particularly, the saturated fatty alcohols are chosen from octyldodecanol, isostearyl alcohol, cetearyl alcohol, 2-hexyldecanol, and also palmityl, myristyl, stearyl and lauryl alcohols, and mixtures thereof.

The unsaturated fatty alcohols contain in their structure at least one double or triple bond, and preferably one or more double bonds. When several double bonds are present, there are preferably 2 or 3 of them, and they may be conjugated or unconjugated. They may optionally comprise in their structure at least one aromatic or non-aromatic ring. Preferably, they are acyclic. More particularly, the unsaturated fatty alcohols are chosen from oleyl alcohol, linoleyl alcohol, linolenyl alcohol and undecylenyl alcohol, and mixtures thereof.

Among the mineral, plant or animal oils that can be used, mention may be made notably: as oils of plant origin, of sweet almond oil, avocado oil, castor oil, olive oil, jojoba oil, sunflower oil, wheatgerm oil, sesame oil, groundnut oil, grapeseed oil, soybean oil, rapeseed oil, safflower oil, coconut oil, corn oil, hazelnut oil, shea butter, palm oil, apricot kernel oil, beauty-leaf oil, evening primrose oil or camelina oil; as oil of animal origin, of perhydrosqualene; as oils of mineral origin, of liquid paraffin and liquid petroleum jelly; and mixtures thereof.

The fatty esters may be esters of monoalcohols or of polyols with monoacids or polyacids, at least one of the alcohols and/or acids including at least one chain of more than 7 carbon atoms. Preferably, the fatty ester according to the invention is chosen from esters of a fatty acid and of a monoalcohol. Preferably, at least one of the alcohols and/or acids is branched. Mention may be made of isopropyl myristate, isopropyl palmitate, isononyl or isostearyl isononanoate, 2-ethylhexyl palmitate, 2-hexyldecyl laurate, 2-octyldecyl palmitate and 2-octyldodecyl myristate, purcellin oil (stearyl octanoate), isopropyl lanolate, and mixtures thereof.

The term “hydrocarbon” means a hydrocarbon composed solely of carbon and hydrogen atoms, which is notably of mineral or plant origin, preferably of plant origin.

As hydrocarbon that can be used in the composition according to the invention, mention may be made of:
linear or branched, optionally cyclic, C₆-C₁₆ alkanes; mention may be made of hexane, undecane, dodecane, tridecane, and isoparaffins, for instance isohexadecane, isododecane and isodecane;
linear or branched hydrocarbons, notably of mineral, animal or synthetic origin with more than 16 carbon atoms, such as volatile or non-volatile liquid paraffins, petroleum jelly, liquid petroleum jelly, polydecenes, hydrogenated polyisobutene such as the product sold under the brand name Parleam® by the company NOF Corporation, and squalane.

More preferentially, the non-silicone fatty substance(s) are chosen from non-silicone oils of mineral origin and C₈-C₃₀ fatty alcohols, and mixtures thereof.

Even more preferentially, the composition according to the invention comprises at least one C₈-C₃₀ fatty alcohol.

Preferably, when the composition according to the invention comprises one or more non-silicone fatty substances, the total content of non-silicone fatty substance(s) ranges from 0.1% to 40% by weight, more preferentially from 1% to 35% by weight, even more preferentially 5% to 30% by weight and even better still from 10% to 25% by weight, relative to the total weight of the composition.

Preferably, when the composition according to the invention comprises one or more non-silicone oils of mineral origin and/or C₈-C₃₀ fatty alcohols, the total content of non-silicone oil(s) of mineral origin and of C₈-C₃₀ fatty alcohol(s) ranges from 0.1% to 40% by weight, more preferentially from 1% to 35% by weight, even more preferentially from 5% to 30% by weight and even better still from 10% to 25% by weight, relative to the total weight of the composition.

More preferentially, when the composition according to the invention comprises one or more C₈-C₃₀ fatty alcohols, the total content of C₈-C₃₀ fatty alcohol(s) ranges from 0.1% to 40% by weight, even more preferentially from 1% to 35% by weight, better still from 5% to 30% by weight and even better still from 10% to 25% by weight, relative to the total weight of the composition.

Preferably, the composition according to the invention comprises water.

Preferably, the total water content is greater than 50% by weight, more preferentially ranges from 50% to 95% by weight, even more preferentially from 55% to 90% by weight and even better still from 60% to 85% by weight, relative to the total weight of the composition.

Advantageously, the aqueous cosmetic composition may also comprise one or more organic solvents.

Use may in particular be made, as examples of organic solvents, of those which are liquid at 25° C. and 1.013×10⁵ Pa, in particular water-soluble, such as C₁-C₇ alcohols and in particular C₁-C₇ aliphatic or aromatic monoalcohols, C₃-C₇ polyols and C₃-C₇ polyol ethers, which can thus be employed as a mixture with water. Mention may most particularly be made of ethanol and isopropanol, and mixtures thereof.

The composition according to the invention may optionally also comprise one or more adjuvants, preferably chosen from anionic, cationic, non-ionic, amphoteric and zwitterionic polymers and/or mixtures thereof, mineral or organic thickeners, antioxidants, penetration agents, fragrances, buffers, dispersants, modified or unmodified, volatile or non-volatile silicones, film-forming agents, preservatives and opacifiers, and mixtures thereof.

Preferably, when the above adjuvant(s) are present in the composition, the adjuvant(s) are generally present in an amount, for each of them, of between 0.01% and 20% by weight, relative to the weight of the composition according to the invention.

The composition according to the invention may optionally also comprise one or more sequestrants, preferably chosen from diethylenetriaminepentaacetic acid (DTPA) and salts thereof, diethylenediaminetetraacetic acid (EDTA) and salts thereof, ethylenediaminedisuccinic acid (EDDS) and salts thereof, etidronic acid and salts thereof, N,N-dicarboxymethylglutamic acid and salts thereof (GLDA), and mixtures thereof.

Of course, those skilled in the art will take care to choose this or these optional additional compound(s) so that the advantageous properties intrinsically associated with the composition according to the invention are not, or not substantially, detrimentally affected by the envisioned addition(s).

The pH of the composition according to the invention is preferably between 3 and 12, more preferentially between 5 and 11.

The pH may be adjusted to the desired value by means of acidifying or alkalinizing agents, or alternatively using buffer systems.

Mention may be made, among the acidifying agents, by way of example, of inorganic or organic acids, such as hydrochloric acid, orthophosphoric acid, sulfuric acid, carboxylic acids, such as acetic acid, tartaric acid, citric acid or lactic acid, or sulfonic acids.

Among the basifying agents, examples that may be mentioned include aqueous ammonia, alkali metal carbonates or bicarbonates, (C₁-C₆)alkanolamines, such as mono-, di- and triethanolamines and derivatives thereof, sodium hydroxide, potassium hydroxide and the compounds of formula (VI) below:

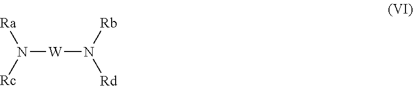

in which W is a (C₁-C₁₀)alkylene group, such as a propylene group, optionally substituted by one or more hydroxyl groups, and Ra, Rb, Rc and Rd, which are identical or different, represent a hydrogen atom or a C₁-C₄ alkyl or C₁-C₄ hydroxyalkyl radical.

The composition according to the invention may be in various forms, such as in the form of liquids, creams or gels, or in any other form that is suitable for dyeing keratin fibres, in particular human keratin fibres and notably the hair.

A subject of the invention is also a process for dyeing keratin fibres, in particular human keratin fibres such as hair, comprising at least one step of applying to said fibres a composition according to the invention, as described above.

Preferably, the process according to the invention also comprises at least one step of applying to the keratin fibres an oxidizing composition, distinct from the dyeing composition according the invention, comprising one or more chemical oxidizing agents.

For the purposes of the present invention, the term “chemical oxidizing agent” means oxidizing agents other than atmospheric oxygen.

Preferably, the chemical oxidizing agent(s) are chosen from hydrogen peroxide, urea peroxide, alkali metal bromates, persalts such as perborates and persulfates, in particular sodium persulfate, potassium persulfate and ammonium persulfate, peracids and oxidase enzymes (with the optional cofactors thereof) such as peroxidases, 2-electron oxidoreductases such as uricases and 4-electron oxygenases such as laccases, and mixtures thereof; preferentially, the chemical oxidizing agent(s) are chosen from hydrogen peroxide, persalts, and mixtures thereof.

More preferentially, the oxidizing composition comprises hydrogen peroxide.

Preferably, the total content of chemical oxidizing agent(s) present in the oxidizing composition is between 1% and 50% by weight, more preferentially between 3% and 30% by weight and even more preferentially between 5% and 20% by weight, relative to the total weight of the oxidizing composition.

Preferably, the total content of hydrogen peroxide present in the oxidizing composition is between 1% and 50% by weight, more preferentially between 3% and 30% by weight and even more preferentially between 5% and 20% by weight, relative to the total weight of the oxidizing composition.

The step of applying the oxidizing composition to the keratin fibres can be carried out before, during or after the implementation of the step of applying the dyeing composition to the keratin fibres.

Preferably, the oxidizing composition is extemporaneously mixed with the dyeing composition according to the invention, then the mixture obtained is applied to the keratin fibres.

According to a preferred embodiment of the invention, when the oxidizing composition is extemporaneously mixed with the dyeing composition according to the invention, the pH of the mixture obtained is preferably between 8 and 11, more preferentially between 9 and 10.7.

The pH may be adjusted to the desired value by means of acidifying or alkalinizing agents, or alternatively using buffer systems, as defined above.

A subject of the invention is also the use of at least one oxidation base (i) chosen from 2-γ-hydroxypropyl-para-phenylenediamine, salts thereof, solvates thereof, and mixtures thereof, in combination with at least one phosphoric surfactant of formula (I), as described above, for dyeing keratin fibres, in particular human keratin fibres such as the hair.

The examples which follow serve to illustrate the invention without, however, exhibiting a limiting nature.

### EXAMPLES

The compositions A (invention), B and C (comparative compositions) are prepared from the ingredients shown in Table 1 below, the amounts of which are expressed, unless otherwise indicated, as weight percentages of active material (AM).

TABLE 1

| | Compo- | Compo- | Compo- |
|---|---|---|---|
| | sition A | sition B | sition C |
| Ingredients | Invention | Comparative | Comparative |
| 2-γ-Hydroxypropyl-p- | 1.14 mmol | — | 1.14 mmol |
| phenylenediamine | | | |
| 2-Methoxymethyl-p- | — | 1.14 mmol | — |
| phenylenediamine | | | |
| 2-Methyl-5- | 0.002 | 0.002 | 0.002 |
| hydroxyethylaminophenol | | | |
| 4-Amino-2- | 0.002 | 0.002 | 0.002 |
| hydroxytoluene | | | |
| 2,4- | 0.006 | 0.006 | 0.006 |
| Diaminophenoxyethanol | | | |
| hydrochloride | | | |
| Resorcinol | 0.14 | 0.14 | 0.14 |
| m-Aminophenol | 0.005 | 0.005 | 0.005 |
| p-Aminophenol | 0.02 | 0.02 | 0.02 |
| Ceteth-10 phosphate | 0.7 | 0.7 | — |
| Sodium lauryl sulfate | — | — | 0.7 |
| Cetearyl alcohol | 11.1 | 11.1 | 11.1 |
| Liquid paraffin | 9 | 9 | 9 |
| Steareth-2 | 1.7 | 1.7 | 1.7 |
| PPG-15 stearyl ether | 1.7 | 1.7 | 1.7 |
| Steareth-20 | 1.1 | 1.1 | 1.1 |
| PEG-20 | 4 | 4 | 4 |
| 2-Oleamido-1,3- | 0.01 | 0.01 | 0.01 |
| octadecanediol | | | |
| Preservatives and | q.s. | q.s. | q.s. |
| pH agents | | | |
| Water | qs 100 | qs 100 | qs 100 |

The oxidizing composition D was prepared from the ingredients indicated in Table 2 below, the amounts of which are expressed as weight percentages of active material (AM).

TABLE 2

| Ingredients | Composition D |
|---|---|
| Hydrogen peroxide | 5.85 |
| Beheneth-10 | 0.5 |
| Ceteareth-33 | 1.7 |
| Cetearyl alcohol | 4.4 |
| Liquid paraffin | 0.8 |
| Polyquaternium-6 | 0.2 |
| Preservatives | q.s. |
| Water | qs 100 |

Protocol:

At the time of use, each of the compositions A to C is mixed with the oxidizing composition D according to a 1:1 weight ratio.

Each of the mixtures M_{(A+D)}, M_{(B+D)} et M_{(C+D)} is then applied to a lock of hair containing 90% natural white hair (NW) and a lock of permanent-waved 90% natural white hair (PWNW) in a proportion of 5 g of mixture per 1 g of hair.

After a leave-on time of 30 min on a hot plate at 27° C., each lock of hair is rinsed, washed with L'Oréal Professionnel Pro Classic universal concentrated shampoo, diluted to 10%, and dried.

Evaluation:

The colorimetric measurements were performed on each of the treated locks using a Konica Minolta CM-3600A spectrocolorimeter (illuminant D65, angle 10°, specular component included) in the CIELab system.

In this system, L* represents the lightness: the lower the value of L*, the deeper, more powerful and more intense the colouring obtained. The chromaticity is measured by the values a* and b*, a* representing the green/red colour axis and b* the blue/yellow colour axis.

The selectivity is represented by the colour difference ΔE between the locks of dyed natural non-permanent-waved (NW) hair and the dyed permanent-waved locks (PWNW).

ΔE=\sqrt{(L*−L₀*)²+(a*−a₀*)²+(b*−b₀*)²}

In this equation, L*, a* and b* represent the values measured on the locks of dyed non-permanent-waved natural (NW) hair, and L₀*, a₀* and b₀* represent the values measured on the locks of dyed permanent-waved (PWNW) hair. The lower the value of ΔE, the lower and therefore better the selectivity (uniform dyeing).

The results obtained are grouped together in Table 3 below:

TABLE 3

| | Type of hair | L* | a* | b* | ΔE |
|---|---|---|---|---|---|
| Mixture M_{A+D} | NW | 48.46 | 3.19 | 12.11 | 1.16 |
| (invention) | PWNW | 47.31 | 3.17 | 11.99 | |
| Mixture M_{B+D} | NW | 49.77 | 3.00 | 11.70 | 7.24 |
| (comparative) | PWNW | 42.62 | 4.10 | 11.94 | |
| Mixture M_{C+D} | NW | 48.39 | 2.92 | 12.30 | 8.96 |
| (comparative) | PWNW | 39.50 | 3.83 | 11.70 | |

The colouring obtained with the mixture M_{A+D} according to the invention has ΔE values lower than that of the colourings obtained with the comparative mixtures M_{B+D} and M_{C+D}.

It is thus observed that the selectivity of the colouring obtained with the composition according to the invention is better than the selectivity of the colourings obtained with the comparative compositions.

The colourings obtained with the composition according to the invention are therefore more homogeneous than the colourings obtained with the comparative compositions.

1. Dyeing composition comprising:
(i) at least one oxidation base chosen from 2-γ-hydroxypropyl-para-phenylenediamine, salts thereof, solvates thereof, and mixtures thereof, and
(ii) at least one phosphoric surfactant chosen from phosphoric surfactants having the formula (I) below: 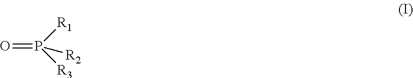
in which:
R₁, R₂ and R₃, which may be identical or different, represent a group chosen from:
a group —OM, in which M represents a hydrogen atom or an alkali metal;
a group —OR₄, in which R₄ represents a linear or branched C₁₂-C₄₀ alkyl group, a linear or branched C₂-C₄₀ alkenyl group, a C₃-C₄₀ cyclic alkyl group, a C₃-C₄₀ cyclic alkenyl group, a C₅-C₄₀ aromatic group or a C₆-C₄₀ aralkyl group; and
an oxyalkylene group —(OCH₂CH₂)ₙ(OCH₂CH(CH₃))ₘOR₄ in which R₄ is defined as above, n represents an integer ranging from 1 to 50 and m represents an integer ranging from 0 to 50; and
given that at least one of the groups R₁, R₂ and R₃ is a group —OM and that at least one of the groups R₁, R₂ and R₃ is a group —OR₄ or —(OCH₂CH₂)ₙ(OCH₂CH(CH₃))ₘOR₄.

2. Composition according to claim 1, characterized in that the total content of oxidation base(s) (i) ranges from 0.001% to 20% by weight, preferably from 0.005% to 15% by weight, more preferentially from 0.01% to 10% by weight, even more preferentially from 0.05% to 5% by weight, and even better still from 0.1% to 3% by weight, relative to the total weight of the composition.

3. Composition according to claim 1, characterized in that it further comprises one or more additional oxidation bases, other than the oxidation base(s) (i), preferably chosen from para-phenylenediamine, para-toluenediamine, para-aminophenol, 2-β-hydroxyethyl-para-phenylenediamine, N,N-bis(β-hydroxyethyl)-para-phenylenediamine, 4,5-diamino-1-(β-hydroxyethyl)pyrazole, 2,3-diamino-6,7-dihydro-1H,5H-pyrazolo[1,2-a]pyrazol-1-one, 2-β-hydroxyethoxy-3-aminopyrazolo[1,5-a]pyridine, addition salts thereof, solvates thereof, and mixtures thereof.

4. Composition according to claim 1, characterized in that it further comprises one or more oxidation couplers, preferably chosen from meta-phenylenediamines, meta-aminophenols, meta-diphenols, naphthalene-based coupling agents, heterocyclic coupling agents, addition salts thereof, solvates thereof, and mixtures thereof; and more preferentially from 2-amino-5-ethylphenol, hydroxyethyl-3,4-methylenedioxyaniline,1,3-dihydroxybenzene, 1,3-dihydroxy-2-methylbenzene, 3-aminophenol, 6-hydroxybenzomorpholine, 5-N-(β-hydroxyethyl)amino-2-methylphenol, 2,4-diamino-1-(β-hydroxyethyloxy)benzene, 2-methyl-5-aminophenol, 6-hydroxyindole, 4-chloro-1,3-dihydroxybenzene, 2-amino-3-hydroxypyridine, 3-amino-2-chloro-6-methylphenol, α-naphthol, 2-[3-amino-4-methoxyphenyl]amino)ethanol, addition salts thereof, solvates thereof, and mixtures thereof.

5. Composition according to claim 1, characterized in that the phosphoric surfactant(s) are chosen from phosphoric surfactants of formula (I), in which:
R₁, R₂ and R₃, which may be identical or different, represent a group chosen from:
a group —OM, in which M represents a hydrogen atom or an alkali metal, such as Na, Li or K, preferably Na or K;
a group —OR₄, in which R₄ represents a linear or branched C₁₂-C₂₀ alkyl group, preferably a C₁₆ or C₁₈ alkyl group, a linear or branched C₂-C₄₀ alkenyl group, preferably a C₁₂-C₂₀ alkenyl group, more preferentially a C₁₆ or C₁₈ alkenyl group, a C₃-C₄₀ cyclic alkyl group, a C₃-C₄₀ cyclic alkenyl group, a C₅-C₄₀ aromatic group or a C₆-C₄₀ aralkyl group; and
an oxyalkylene group —(OCH₂CH₂)ₙ(OCH₂CH(CH₃))ₘOR₄ in which R₄ is defined as above, n represents an integer ranging from 1 to 50 and m represents an integer ranging from 0 to 50; and
given that at least one of the groups R₁, R₂ and R₃ is a group —OM and that at least one of the groups R₁, R₂ and R₃ is a group —OR₄ or —(OCH₂CH₂)ₙ(OCH₂CH(CH₃))ₘOR₄.

6. Composition according to claim 1, characterized in that the phosphoric surfactant(s) are chosen from ceteth-10 phosphate, dicetyl phosphate, ceteth-20 phosphate, oleth-5 phosphate and dioleyl phosphate, salts thereof, and mixtures thereof; preferably from ceteth-10 phosphate, ceteth-20 phosphate and oleth-5 phosphate, salts thereof, and mixtures thereof; and even better still from ceteth-10 phosphate, salts thereof, and mixtures thereof.

7. Composition according to claim 1, characterized in that the total content of phosphoric surfactant(s) of formula (I) ranges from 0.01% to 15% by weight, preferably from 0.05% to 10% by weight, more preferentially from 0.075% to 5% by weight, even more preferentially from 0.1% to 2% by weight, and even better still from 0.5% to 1% by weight, relative to the total weight of the composition.

8. Composition according to claim 1, characterized in that the weight ratio of the total content of phosphoric surfactant(s) of formula (I) on the one hand, to the total content of 2-γ-hydroxypropyl-para-phenylenediamine on the other hand, is greater than or equal to 1, preferentially greater than or equal to 1.5, more preferentially greater than or equal to 2, even more preferentially greater than or equal to 2.5.

9. Composition according to claim 1, characterized in that it further comprises one or more non-silicone fatty substances, preferably chosen from non-silicone oils of mineral origin, C₈-C₃₀ fatty alcohols, and mixtures thereof, and more preferentially from C₈-C₃₀ fatty alcohols.

10. Composition according to claim 9, characterized in that the total content of non-silicone fatty substance(s) ranges from 0.1% to 40% by weight, preferably from 1% to 35% by weight, more preferentially from 5% to 30% by weight, and even more preferentially from 10% to 25% by weight, relative to the total weight of the composition.

11. Composition according to claim 1, characterized in that it further comprises one or more additional surfactants other than the phosphoric surfactants, preferably one or more non-ionic additional surfactants other than the phosphoric surfactants.

12. Composition according to claim 11, characterized in that the total content of additional surfactant(s) other than the phosphoric surfactants ranges from 0.01% to 20% by weight, preferably from 0.5% to 15% by weight, more preferentially from 1% to 15% by weight, and even more preferentially from 3% to 10% by weight, relative to the total weight of the composition.

13. Process for dyeing keratin fibres, comprising at least one step of applying to said fibres a composition as defined in claim 1.

14. Process according to claim 13, further comprising at least one step of applying to said fibres an oxidizing composition comprising one or more chemical oxidizing agents, preferably hydrogen peroxide.

15. Use of at least one oxidation base (i) chosen from 2-γ-hydroxypropyl-para-phenylenediamine, salts thereof, solvates thereof, and mixtures thereof, in combination with at least one phosphoric surfactant of formula (I) as defined in claim 1, for dyeing keratin fibres.
